(19) United States
(12) Patent Application Publication
(10) Pub. No.: US 2024/0225503 A9
(43) Pub. Date: Jul. 11, 2024

(54) INFORMATION PROCESSING DEVICE, CONTROL METHOD, AND STORAGE MEDIUM

(71) Applicant: NEC Corporation, Minato-ku, Tokyo (JP)

(72) Inventor: Azusa Furukawa, Tokyo (JP)

(73) Assignee: NEC Corporation, Minato-ku, Tokyo (JP)

(21) Appl. No.: 18/277,726

(22) PCT Filed: Mar. 24, 2021
(86) PCT No.: PCT/JP2021/012274

§ 371 Date: Aug. 17, 2023

(51) Int. Cl. A61B 5/16; A61B 5/00

(52) U.S. Cl. CPC A61B 5/165; A61B 5/7264

(57) ABSTRACT

An information processing device 1X mainly includes an emotion acquisition means 14X, a deviation tendency identification means 16X, and an output control means 17X. The emotion acquisition means 14X acquires a set of target emotion, which is a target of emotion of an object person, and actual emotion, which is actual emotion of the object person. The deviation tendency identification means 16X identifies a deviation tendency between the target emotion and the actual emotion with respect to a mental state, based on the set of the target emotion and the actual emotion. The output control means 17X outputs information regarding the deviation tendency identified by the deviation tendency identification means 16X.

### TECHNICAL FIELD

The present disclosure relates to a technical field of an information processing device, a control method, and a storage medium configured to perform processing related to estimation of a mental state.

### BACKGROUND

There are known a device or a system for estimating the mental state of an object person. For example, Patent Literature 1 discloses a technique of evaluating an emotion felt by an object person based on both aspects of arousal degree and comfort degree and adjusting the air volume, the temperature, the aroma, and the like of an air conditioner based on the arousal degree data and the comfort degree data.

### SUMMARY

### Problem to be Solved

Patent Literature 1: JP 2019-208576A

### SUMMARY

### Problem to be Solved

Since there is no correct answer of the emotion, the approach to manage and adjust the emotion varies depending on the person. Then, in order to properly manage and adjust his/her emotion, it is insufficient to grasp only the present emotion.

In view of the above-described issues, it is an object of the present disclosure to provide an information processing device, a control method, and a storage medium capable of supporting management and adjustment of the emotion.

### Means for Solving the Problem

In one mode of the information processing device, there is provided an information processing device including:
an emotion acquisition means configured to acquire a set of target emotion, which is a target of emotion of an object person, and actual emotion, which is actual emotion of the object person;
a deviation tendency identification means configured to identify a deviation tendency between the target emotion and the actual emotion with respect to a mental state, based on the set of the target emotion and the actual emotion; and
an output control means configured to output information regarding the deviation tendency.

In one mode of the control method, there is provided a control method executed by a computer, the control method including:
acquiring a set of target emotion, which is a target of emotion of an object person, and actual emotion, which is actual emotion of the object person;
identifying a deviation tendency between the target emotion and the actual emotion with respect to a mental state, based on the set of the target emotion and the actual emotion; and
outputting information regarding the deviation tendency.
   It is noted that examples of “computer” include any kind of electric devices (including a processor included in an electric device) and may be configured by plural electric devices.

In one mode of the storage medium, there is provided a storage medium storing a program executed by a computer, the program causing the computer to:
acquire a set of target emotion, which is a target of emotion of an object person, and actual emotion, which is actual emotion of the object person;
identify a deviation tendency between the target emotion and the actual emotion with respect to a mental state, based on the set of the target emotion and the actual emotion; and
output information regarding the deviation tendency.

### Effect

An example advantage according to the present invention is to suitably output information useful for management and adjustment of the emotion.

### BRIEF DESCRIPTION OF THE DRAWINGS

FIG. 1 shows a schematic configuration of a mental state estimation system according to the first example embodiment.

FIG. 2 is a hardware configuration of the information processing device.

FIG. 3 is an example of a functional block of the information processing device.

FIG. 4A is a first display example of the target emotion input screen image.

FIG. 4B is a second display example of the target emotion input screen image.

FIG. 5A is an example of a data structure of emotion management information in the first embodiment.

FIG. 5B is an example of a data structure of the deviation tendency information in the first embodiment.

FIG. 6A is a diagram showing a deviation vector in the mental state coordinate system.

FIG. 6B is an output example of an emotion assessment report in the first embodiment.

FIG. 7 is an example of a functional block of the information processing device in the second embodiment.

FIG. 8A is a display example of the first event designation screen image.

FIG. 8B is a display example of the second event designation screen image.

FIG. 9A is an example of a data structure of emotion management information in the second embodiment.

FIG. 9B is an example of a data structure of the deviation tendency information in the second embodiment.

FIG. 9C is an example of an emotion assessment report in the second embodiment.

FIG. 10 is an example of a functional block of the information processing device in the third embodiment.

FIG. 11A is an example of a data structure of emotion management information in the third embodiment.

FIG. 11B is an example of a data structure of the deviation tendency information in the third embodiment.

FIG. 11C is an example of an emotion assessment report in the third embodiment.

FIG. 12 is an example of functional blocks of the information processing device in the fourth embodiment.

FIG. 13A is a first example of an emotion assessment report in the fourth embodiment.

FIG. 13B is a second example of an emotion assessment report in the fourth embodiment.

FIG. 14 is an example of a processing flowchart to be executed by the information processing device in the first example embodiment.

FIG. 15 is an example of a system usage scenario by an object person.

FIG. 16 shows a schematic configuration of a mental state estimation system in the second example embodiment.

FIG. 17 is a block diagram of an information processing device according to a third example embodiment.

FIG. 18 is an example of a flowchart to be executed by an information processing device in the third example embodiment.

### EXAMPLE EMBODIMENTS

Hereinafter, example embodiments of an information processing device, a control method, and a storage medium will be described with reference to the drawings.

### First Example Embodiment

### (1) System Configuration

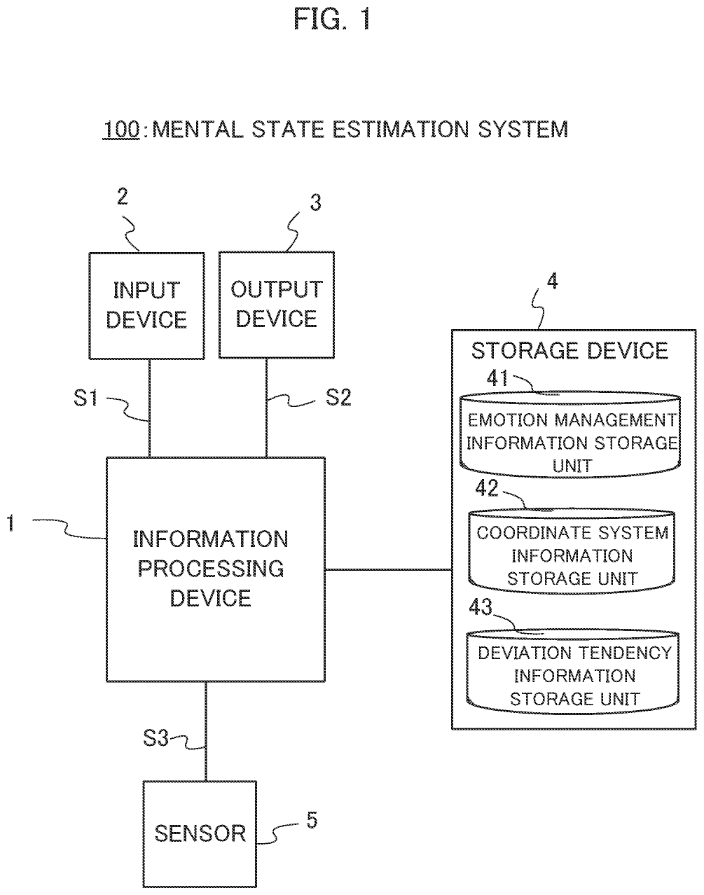

FIG. 1 shows a schematic configuration of a mental state estimation system 100 according to a first example embodiment. The mental state estimation system 100 identifies a tendency of deviation between the actual emotion (also referred to as “actual emotion”) of an object person (subject) and the emotion (also referred to as “target emotion”) to be a target (goal) of the object person and outputs information regarding the identified tendency of deviation. Here, examples of the “object person” include a sports player, an employee whose mental state is managed by the organization, and an individual user.

The mental state estimation system 100 mainly includes an information processing device 1, an input device 2, an output device 3, a storage device 4, and a sensor 5.

The information processing device 1 performs data communication with the input device 2, the output device 3, and the sensor 5 through a communication network or through wireless or wired direct communication. Then, the information processing device 1 identifies the actual emotion and target emotion of the object person and identifies a deviation tendency between the actual emotion and the target emotion, on the basis of the input signal “S1” supplied from the input device 2, the sensor signal “S3” supplied from the sensor 5, and information stored in the storage device 4. The information processing device 1 also generates an output signal “S2” regarding the identified deviation tendency and supplies the generated output signal S2 to the output device 3.

The input device 2 is one or more interfaces that accept manual input (external input) of information regarding each object person. The user who performs input of information using the input device 2 may be the object person itself, or may be a person who manages or supervises the activity of the object person. Examples of the input device 2 include a touch panel, a button, a keyboard, a mouse, a voice input device and any other variety of user input interfaces. The input device 2 supplies the generated input signal S1 to the information processing device 1. The output device 3 displays or outputs predetermined information based on the output signal S2 supplied from the information processing device 1. Examples of the output device 3 include a display, a projector, and a speaker.

The sensor 5 measures the object person's biological data (biological signal) or the like, and supplies the measured biological data or the like to the information processing device 1 as a sensor signal S3. In this instance, the sensor signal S3 may be any biological data (e.g., heartbeat, EEG, amount of perspiration, amount of hormonal secretion, cerebral blood flow, blood pressure, body temperature, electromyogram, electrocardiogram, respiration rate, pulse wave, acceleration) used for stress estimation of the object person. The sensor 5 may be a device that analyzes blood of the object person and outputs the analysis result as a sensor signal S3. The sensor 5 may be a wearable terminal worn by the object person, or may be a camera for photographing the object person or a microphone for generating a voice signal of the object person's utterance.

The storage device 4 is one or more memories stores various types of information necessary for the information processing device 1 to execute the processing. Examples of the storage device 4 may include an external storage device, such as a hard disk, connected to or embedded in the information processing device 1, and a storage medium, such as a flash memory. The storage device 4 may be a server device that performs data communication with the information processing device 1. Further, the storage device 4 may be configured by a plurality of devices.

The storage device 4 functionally includes an emotion management information storage unit 41, a coordinate system information storage unit 42, and a deviation tendency information storage unit 43.

The emotion management information storage unit 41 stores plural sets of the target emotion and the actual emotion of the object person measured or inputted in time series. As will be described later, the information processing device 1 acquires a set of the target emotion and the actual emotion of the object person at constant or undefined intervals and stores the acquired sets of the target emotion and the actual emotion in the emotion management information storage unit 41. The details of the data structure of emotion management information will be described later.

The coordinate system information storage unit 42 stores coordinate system information which is information regarding a coordinate system (also referred to as “mental state coordinate system”) of the mental state capable of representing each emotion by coordinate values. The mental state coordinate system may be any coordinate system representing emotion. For example, the mental state coordinate system may be a coordinate system (coordinate system whose axes are valence of comfort-discomfort and arousal degree) adopted in the Russell's circumplex model or may be a coordinate system (coordinate system whose axes are anxiety level/ease level and exciting degree/frustrating level) adopted in KOKORO scale. Then, for example, the coordinate system information storage unit 42 stores the coordinate system information in the form of table information which indicates correspondence between each possible emotion and the corresponding coordinate value in the mental state coordinate system.

The deviation tendency information storage unit 43 stores deviation tendency information representing a deviation tendency between the target emotion and the actual emotion of the object person, which is identified by the information processing device 1 for each set of the target emotion and the actual emotion. Details of the data structure of deviation tendency information will be described later.

The configuration of the mental state estimation system 100 shown in FIG. 1 is an example, various changes may be made to the configuration. For example, the input device 2 and the output device 3 may be configured integrally. In this case, the input device 2 and the output device 3 may be configured as a tablet type terminal that is integrated with or separate from the information processing device 1. Further, the input device 2 and the sensor 5 may be configured integrally. Further, the information processing device 1 may be configured by a plurality of devices. In this case, the plurality of devices constituting the information processing device 1 performs transmission and reception of information necessary for executing preassigned processing among the plurality of devices. In this case, the information processing device 1 functions as an information processing system.

### (2) Hardware Configuration of Information Processing Device

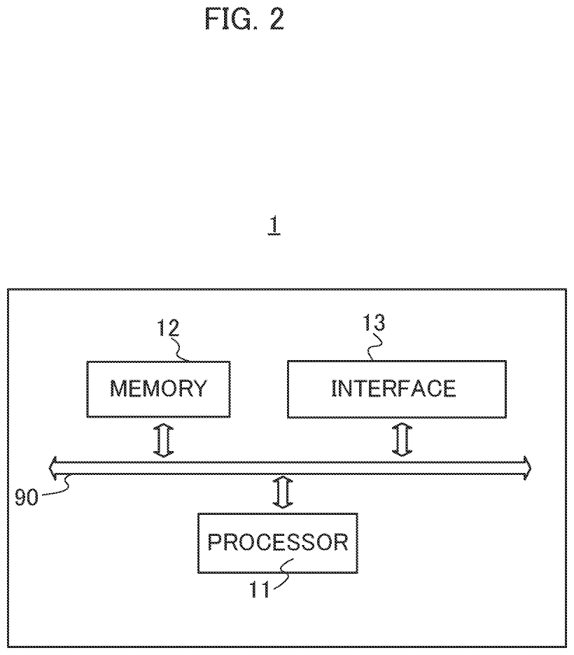

FIG. 2 shows a hardware configuration of the information processing device 1. The information processing device 1 includes a processor 11, a memory 12, and an interface 13 as hardware. The processor 11, memory 12 and interface 13 are connected via a data bus 90 to one another.

The processor 11 functions as a controller (arithmetic unit) configured to control the entire information processing unit 1 by executing a program stored in the memory 12. Examples of the processor 11 include a CPU (Central Processing Unit), a GPU (Graphics Processing Unit), and a TPU (Tensor Processing Unit). The processor 11 may be configured by a plurality of processors. The processor 11 is an example of a computer.

The memory 12 is configured by a variety of volatile and non-volatile memories, such as a RAM (Random Access Memory), a ROM (Read Only Memory), and a flash memory. Further, a program for executing a process executed by the information processing device 1 is stored in the memory 12. A part of the information stored in the memory 12 may be stored by one or more external storage devices that can communicate with the information processing device 1, or may be stored by a storage medium detachable to the information processing device 1.

The interface 13 is one or more interfaces for electrically connecting the information processing device 1 to other devices. Examples of these interfaces may include a wireless interface, such as a network adapter, for transmitting and receiving data to and from other devices wirelessly, and a hardware interface, such as a cable, for connecting to other devices.

The hardware configuration of the information processing device 1 is not limited to the configuration shown in FIG. 2. For example, the information processing device 1 may include at least one of the input device 2 and the output device 3. Further, the information processing device 1 may be connected to or incorporate a sound output device such as a speaker.

### (3) Details of the Processing Performed by the Information Processing Unit

Next, a detailed description of the process performed by the information processing device 1 will be described. In the following, the specific embodiments (first embodiment to fourth embodiment) relating to the identification and output of the deviation tendency between the target emotion and the actual emotion will be described in order.

### (3-1) First Aspect

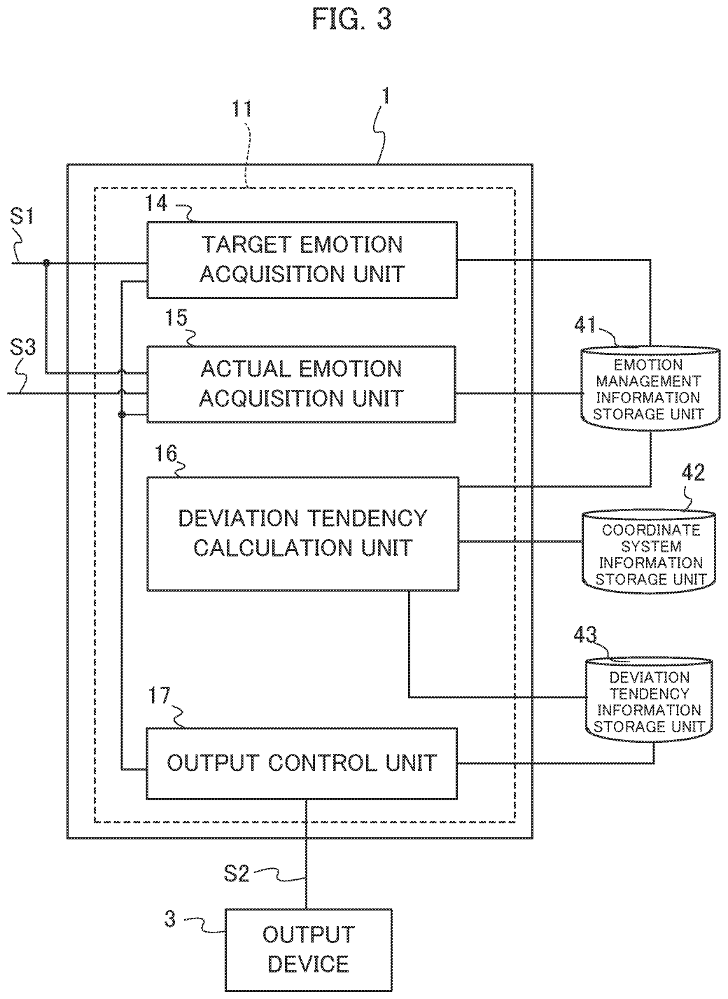

FIG. 3 is an example of a functional block of the information processing device 1 in the first embodiment. The processor 11 of the information processing device 1 according to the first embodiment functionally includes a target emotion acquisition unit 14, an actual emotion acquisition unit 15, a deviation tendency calculation unit 16, and an output control unit 17. In FIG. 3, blocks to exchange data with each other are connected by a solid line, but the combination of blocks to exchange data with each other is not limited to FIG. 3. The same applies to the drawings of other functional blocks described below.

The target emotion acquisition unit 14 acquires the target emotion of the object person based on the input signal S1. In this case, for example, the target emotion acquisition unit 14 displays the target emotion input screen image to be described later on the output device 3 and receives an input to specify the target emotion on the target emotion input screen image.

The actual emotion acquisition unit 15 acquires the actual emotion based on at least one of the input signal S1 and the sensor signal S3. For example, the actual emotion acquisition unit 15 acquires, through the interface 13, the sensor signal S3 that is an image supplied from the camera that photographs the object person and analyzes the facial expression of the object person from the acquired image to thereby recognize the emotion of the object person. In this case, for example, the actual emotion acquisition unit 15 performs the above-described recognition using a model configured to infer the facial expression of a person in an image when the image is inputted thereto. In this case, in the storage device 4 or the memory 12, parameters of the above-described deep learning model which are trained in advance are stored. In another example, the actual emotion acquisition unit 15 may estimate the actual emotion of the object person based on the voice data of the object person. In this instance, the actual emotion acquisition unit 15 acquires the voice data generated by a voice input device as the input signal S1 and analyzes the tone of the utterance of the object person or the uttered word or the like based on the acquired voice data to thereby estimate the emotion of the object person. In this case, the storage device 4 or the memory 12 previously stores the information necessary for analyzing the voice data. In yet another example, the actual emotion acquisition unit 15 displays the actual emotion input screen image to be described later to the output device 3 and receives an input specifying the actual emotion in the actual emotion input screen image.

The target emotion acquisition unit 14 and the actual emotion acquisition unit 15 store the sets of the acquired target emotion and actual emotion in the emotion management information storage unit 41. Each of the target emotion and the actual emotion stored in the emotion control information storage unit 41 may be represented, for example, by ID (emotion ID) which is assigned to each possible emotion in advance, or may be represented by the coordinate value in the mental state coordinate system. In the latter case, for example, the target emotion acquisition unit 14 or the actual emotion acquisition unit 15 executes processing for calculating the coordinate values of the target emotion and the actual emotion in the mental state coordinate system, on behalf of the deviation tendency calculation unit 16 to be described later.

The deviation tendency calculation unit 16 calculates the deviation tendency of emotion based on a set of the target emotion and actual emotion stored in the emotion management information storage unit 41. In this case, the deviation tendency calculation unit 16 converts the target emotion and the actual emotion into the coordinate values in the mental state coordinate system based on the coordinate system information stored in the coordinate system information storage unit 42, and calculates a vector (also referred to as “deviation vector”), in the mental state coordinate system, whose start point is the coordinate value of the target emotion and whose end point is the coordinate value of the actual emotion. Then, the deviation tendency calculation unit 16 stores the information regarding the calculated deviation vector as the deviation tendency information in the deviation tendency information storage unit 43.

The output control unit 17 performs control to output the report information (also referred to as “emotion assessment report”) on the deviation tendency between the target emotion and the actual emotion of the object person based on the deviation tendency information stored in the deviation tendency information storage unit 43 at a predetermined timing. Here, the emotion assessment report is generated based on deviation tendency information corresponding to one or more sets of the target emotion and actual emotion, and examples of the emotion assessment report include a report on the daily emotion of the object person and a report on the medium to long-term emotion of the object person. The output control unit 17 generates an output signal S2 at a predetermined timing and supplies the output signal S2 to the output device 3 through the interface 13 to thereby cause the output device 3 to display an emotion assessment report or output by audio. Examples of the predetermined timing include a timing at a predetermined time, a timing at predetermined time intervals, and any timing requested by the user.

Each component of the target emotion acquisition unit 14, the actual emotion acquisition unit 15, the deviation tendency calculation unit 16, and the output control unit 17 described in FIG. 3 can be realized by the processor 11 executing a program, for example. Additionally, the necessary programs may be recorded on any non-volatile storage medium and installed as necessary to realize each component. It should be noted that at least a portion of each of these components may be implemented by any combination of hardware, firmware, and software, without being limited to being implemented by software based on a program. At least some of these components may also be implemented using user programmable integrated circuit such as, for example, a FPGA (Field-Programmable Gate Array) and a microcontroller. In this case, the integrated circuit may be used to realize a program functioning as each of the above components. Further, at least a part of the components may be configured by ASSP (Application Specific Standard Produce), ASIC (Application Specific Integrated Circuit) or a quantum processor (quantum computer control chip). Thus, each of the above-described components may be realized by various hardware. Furthermore, each of these components may be implemented by cooperation of a plurality of computers, for example, using cloud computing technology. The above is true for other example embodiments described later.

Next, a specific example embodiment of obtaining a target emotion based on the input signal S1 in the first embodiment will be described.

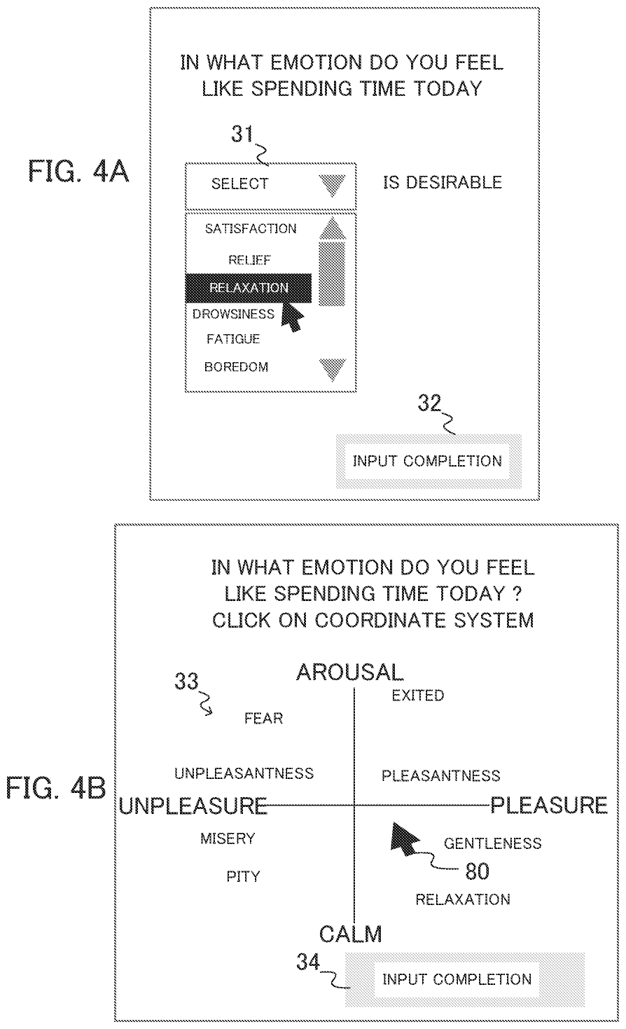

FIG. 4A shows a first display example of the target emotion input screen image. The target emotion input screen image according to the first display example includes a target emotion input field 31 and an input completion button 32. The output control unit 17 generates, based on the instructions from the target emotion acquisition unit 14, an output signal S2 for outputting the target emotion input screen image shown in FIG. 4A, and supplies the generated output signal S2 to the output device 3. Here, as an example, the target emotion input field 31 is an input field in a pull-down menu format. Then, if the input completion button 32 is selected, the output control unit 17 acquires the input signal S1 indicating the target emotion inputted in the target emotion input field 31 at the time when the input completion button 32 is selected, and supplies the target emotion to the target emotion acquisition unit 14.

FIG. 4B shows a second display example of the target emotion input screen image. The target emotion input screen image according to the second display example includes a coordinate system display area 33, and an input completion button 34. The coordinate system display area 33 illustrates the mental state coordinate system used by the information processing device 1. Here, the mental state coordinate system is a coordinate system adopted in Russell's circumplex model, and the positions of some typical emotions are specified on the mental state coordinate system. The output control unit 17 receives the position designation by the mouse pointer 80 on the coordinate system display area 33. When the output control unit 17 detects that the input completion button 32 has been selected, it acquires an input signal S1 representing the position specified by the mouse pointer 80 on the coordinate system display area 33 and supplies the input signal S1 to the target emotion acquisition unit 14. In this instance, the target emotion acquisition unit 14 acquires the coordinate value on the mental state coordinate system corresponding to the position indicated by the input signal S1 as the coordinate value of the target emotion on the mental state coordinate system.

In this way, the target emotion acquisition unit 14 can suitably acquire the target emotion. The actual emotion acquisition unit 15 may also acquire the actual emotion based on the user input in the same manner as the target emotion. In this case, the actual emotion acquisition unit 15 causes the output control unit 17 to output an actual emotion input screen image having an input field of the actual emotion or a display area of the mental state coordinate system on the output device 3 and receives the user input of the information regarding the actual emotion.

Next, the data structure of the emotion management information and the deviation tendency information in the first embodiment will be described.

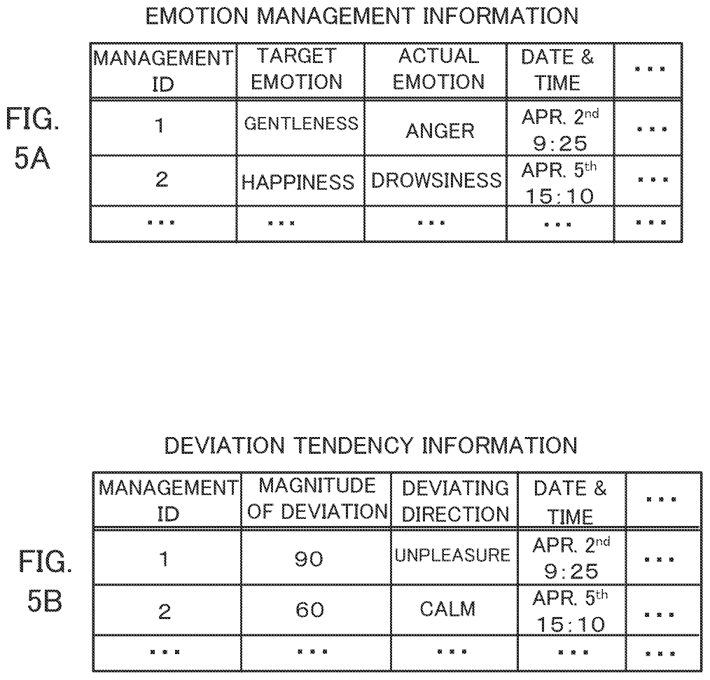

FIG. 5A is an example of a data structure of emotion management information in the first embodiment. As shown in FIG. 5A, emotion management information in the first embodiment includes items “management ID”, “target emotion”, “actual emotion” and “date and time”.

The “management ID” is an ID that is assigned to each set of the target emotion and the actual emotion, and is a serial number according to the generation order of the sets of the target emotion and the actual emotion, as an example. The “target emotion” is the target emotion acquired by the target emotion acquisition unit 14, and the “actual emotion” is the actual emotion acquired by the actual emotion acquisition unit 15. In the “target emotion” and the “actual emotion”, the emotion ID or the like for identifying the emotion may be registered instead of a word representing the emotion being registered therein. The “date and time” indicates the date and time that the object person had the corresponding actual emotion (in other words, the date and time when the actual emotion has occurred).

Here, the update of emotion management information will be supplementally described. For example, when the actual emotion is measured, the information processing device 1 adds, to the emotion management information, a record including “actual emotion” indicating the measured actual emotion, a “date and time” indicating the date and time when the actual emotion has occurred, and “target emotion” indicating the target emotion set at the date and time. The target emotion of a day may be set at a time at the beginning of the day, and the actual emotion at a certain timing may be inputted by a user with some time lag if the actual emotion is manually inputted.

The emotion management information may have various items, in addition to the items described above. For example, when the information processing device 1 handles emotions of plural object persons, the emotion control information may further include the object person ID corresponding to the actual emotion and the target emotion.

FIG. 5B is an example of a data structure of the deviation tendency information in the first embodiment. As shown in FIG. 5B, the deviation tendency data includes items “management ID,” “magnitude of deviation”, “deviating direction”, and “date and time”.

The “management ID” indicates an ID for identifying each set of the target emotion and actual emotion. The “magnitude of deviation” indicates the magnitude of the deviation vector calculated based on the corresponding set of the target emotion and actual emotion. The “deviating direction” indicates the direction of the deviation vector. Here, the direction of the deviation vector indicates the direction of the coordinate axis closest to the deviation vector. Here, the mental state coordinate system is assumed to be a coordinate system whose horizontal axis is the valence of “comfort”-“discomfort” and whose vertical axis is “arousal”-“calm”, which is adopted in the Russell's circumplex model. The “date and time” indicates the date and time that the object person had the corresponding actual emotion.

The deviation tendency information may have various items in addition to the items shown in FIG. 5B. For example, when the information processing device 1 handles a plurality of object persons, the deviation tendency information may further include the object person ID. In another example, the deviation tendency information may include an item representing the coordinate value (i.e., the relative coordinate value of actual emotion when the coordinate value of the target emotion is set as the origin) of the deviation vector in place of or in addition to the “deviation size” and the “deviating direction”.

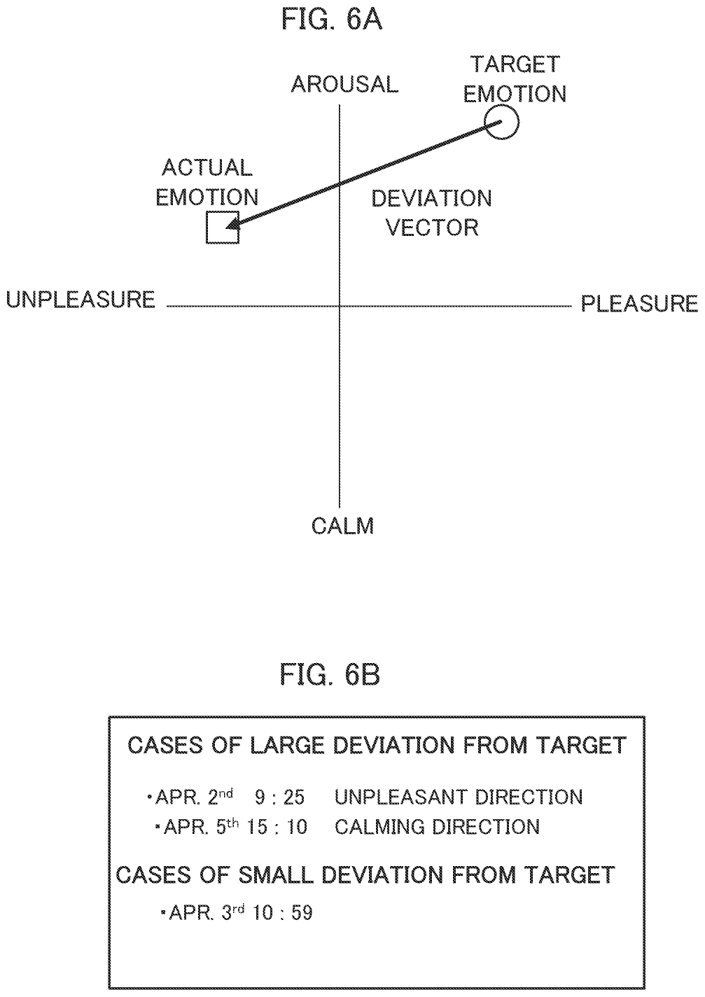

FIG. 6A is a diagram showing a deviation vector corresponding to the management ID “1” shown in FIGS. 5A and 5B. Here, the mental state coordinate system is a coordinate system whose horizontal axis is the valence of “comfort”-“discomfort” and whose vertical axis is “arousal”-“calm”, which is adopted in the Russell's circumplex model, wherein the positions of the actual emotion and the target emotion are indicated clearly. In this case, the actual emotion deviates from the target emotion, and the deviation vector is oriented in the direction of “unpleasure” and “calm”, among which the element of “unpleasure” is especially large.

FIG. 6B is an example of an emotion assessment report outputted by the output control unit 17 to the output device 3 in the first example embodiment.

In the emotion assessment report shown in FIG. 6B, the output control unit 17 outputs the contents of records of deviation tendency information corresponding to plural sets of the target emotion and actual emotion including the management ID “1” and “2” measured in the most recent predetermined period (e.g., in one week). Here, the output control unit 17 outputs the contents of the records of the deviation tendency information in which the size of the deviation vector is equal to or larger than a predetermined threshold value as “cases of large deviation from target”, and outputs the contents of the record of the deviation tendency information in which the size of the deviation vector is smaller than the predetermined threshold value as “cases of small deviation from target”. Thus, the output control unit 17 determines the output embodiment of the emotion assessment report on the basis of the magnitude of the deviation vector.

For example, the output control unit 17 determines that the magnitude of the deviation vector is equal to or larger than the predetermined threshold value for each record of the deviation tendency information corresponding to the management ID “1” and “2”, and recognizes them as “cases of large deviation from target”. Then, the output control unit 17 outputs the contents (here, the date and time and deviation direction) of the respective records of the deviation tendency information corresponding to the management ID “1” and “2” as “cases of large deviation from target”.

In this way, the output control unit 17 according to the first embodiment outputs the emotion assessment report as shown in FIG. 6B, and notifies the object person or the manager of the deviation between the target emotion and the actual emotion of the object person, suitably supporting the recognition and management adjustment of the emotion of the object person. Thereby, it is possible to stimulate the object person's growth in EQ (Emotional Intelligence Quotient) plane, and lead to the productivity improvement of the work. The output control unit 17 may display the emotion assessment report shown in FIG. 6B on the output device 3 as it is, or may output, by audio, the information corresponding to the emotion assessment report to the output device 3. In yet another example, the output control unit 17 may generate a file corresponding to the emotion assessment report shown in FIG. 6B.

### (3-2) Second Embodiment

In the second embodiment, in addition to the processing in the first embodiment, the information processing device 1 further acquires the event information regarding the object person and determines the output mode of the emotion assessment report based on the event information.

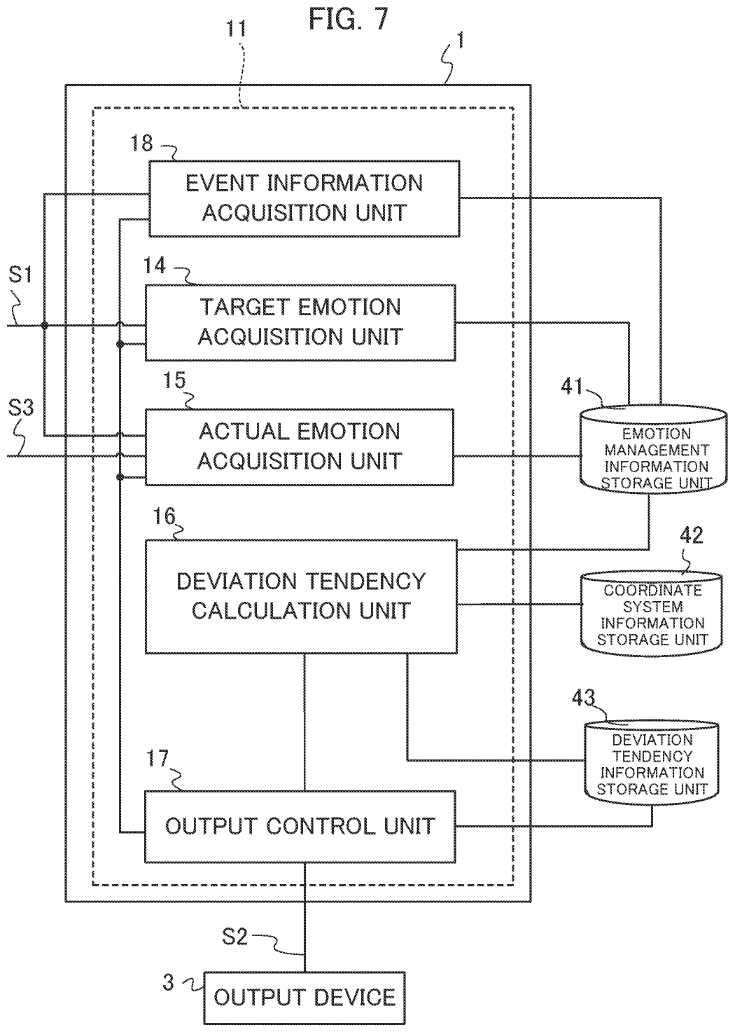

FIG. 7 is an example of functional blocks of the information processing device 1 according to the second embodiment. The processor 11 of the information processing device 1 according to the second embodiment functionally includes a target emotion acquisition unit 14, an emotion acquisition unit 15, a deviation tendency calculation unit 16, an output control unit 17, and an event information acquisition unit 18.

The target emotion acquisition unit 14 and the actual emotion acquisition unit 15 acquire the target emotion and the actual emotion of the object person before, after or during an event related to the object person. The target emotion in this case may be individually set for each event, or may be set weekly or daily regardless of the event. The target emotion and the actual emotion are stored in the emotion management information storage unit 41 in association with the event information detected by the event information acquisition unit 18. The deviation tendency calculation unit 16 calculates the deviation vector for each set of the target emotion and the actual emotion, and adds a record corresponding to the calculated deviation vector to the deviation tendency information stored in the deviation tendency information storage unit 43. The output control unit 17 determines the output mode of the emotion assessment report based on the event information and outputs the emotion assessment report according to the determined output mode. The output control unit 17 controls the output device 3 to output each input screen image regarding the target emotion, the actual emotion, and the event, on the basis of instructions from the target emotion acquisition unit 14, the actual emotion acquisition unit 15, and the event information acquisition unit 18.

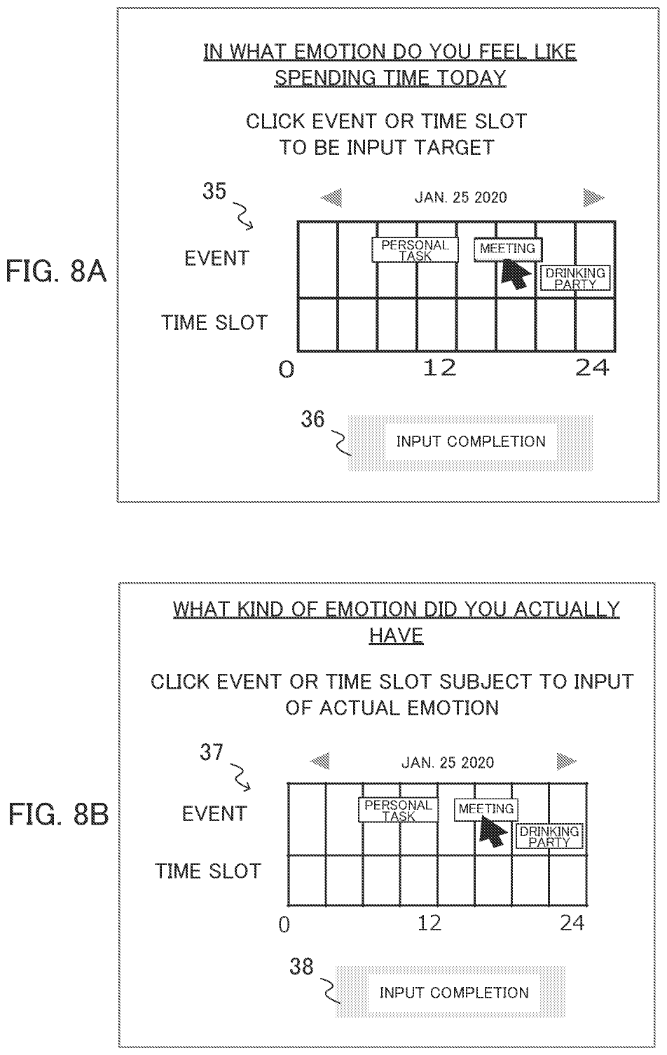

The event information acquisition unit 18 acquires the event information regarding the event of the object person. Examples of the event information include information regarding the content (details) of the event and information regarding the date and time (time slot) of the event. In this case, for example, based on the input signal S1, the event information acquisition unit 18 acquires the event information representing the event corresponding to the set of the target emotion and the actual emotion acquired by the target emotion acquisition unit 14 and the actual emotion acquisition unit 15. In this case, the event information acquisition unit 18 may acquire the schedule information regarding the object person from a system or the like that manages the schedule of the object person thereby to identify the event that corresponds to the set of the target emotion and the actual emotion, based on the acquired schedule information. This specific example will be described with reference to FIGS. 8A and 8B.

FIG. 8A is a display example of the first event designation screen image, which is a screen image in which the user specifies an event to be associated with the target emotion to be inputted. The first event designation screen image shown in FIG. 8A has a personal schedule display area 35, and an input completion button 36. In this case, the output control unit 17 acquires the schedule information regarding the object person from the storage device 4 or other schedule management device, and displays the schedule of the object person in the personal schedule display area 35. In FIG. 8A, as an example, the output control unit 17 displays the schedule of the object person on Jan. 25, 2020, and displays the registered events “personal task”, “meeting”, and “drinking party” in association with the date and time of each event.

Then, in the personal schedule display area 35 of the first event designation screen image, the output control unit 17 receives an input specifying an event or time slot to be associated with the target emotion to be inputted. Then, if the output control unit 17 detects that the input completion button 36 has been selected, the output control unit 17 detects the selected event or an event corresponding to the selected time slot in the personal schedule display area 35 as an event to be associated with the target emotion to be subsequently inputted. Thereafter, the output control unit 17 displays the target emotion input screen image (see FIG. 4A or FIG. 4B) described in the first embodiment and further receives an input specifying the target emotion.

FIG. 8B is a display example of a second event designation screen image that is a screen image in which a user specifies an event to be associated with the actual emotion to be inputted. The second event designation screen image shown in FIG. 8B is provided with a personal schedule display area 37 and an input completion button 38. In this case, the output control unit 17 acquires the schedule information regarding the object person from the storage device 4 or any other schedule management device, and displays the schedule regarding the object person in the personal schedule display area 37 based on the schedule information. Then, in the second event designation screen image, the output control unit 17 receives an input specifying an event or time slot to be associated with the actual emotion to be inputted in the personal schedule display area 37. Then, if the output control unit 17 detects that the input completion button 36 is selected, it identifies the event corresponding to the selected event or the selected time slot in the personal schedule display area 37 as an event to be associated with the actual emotion to be subsequently inputted. Thereafter, the output control unit 17 displays the actual emotion input screen image described in the first embodiment and further receives an input that specifies the actual emotion.

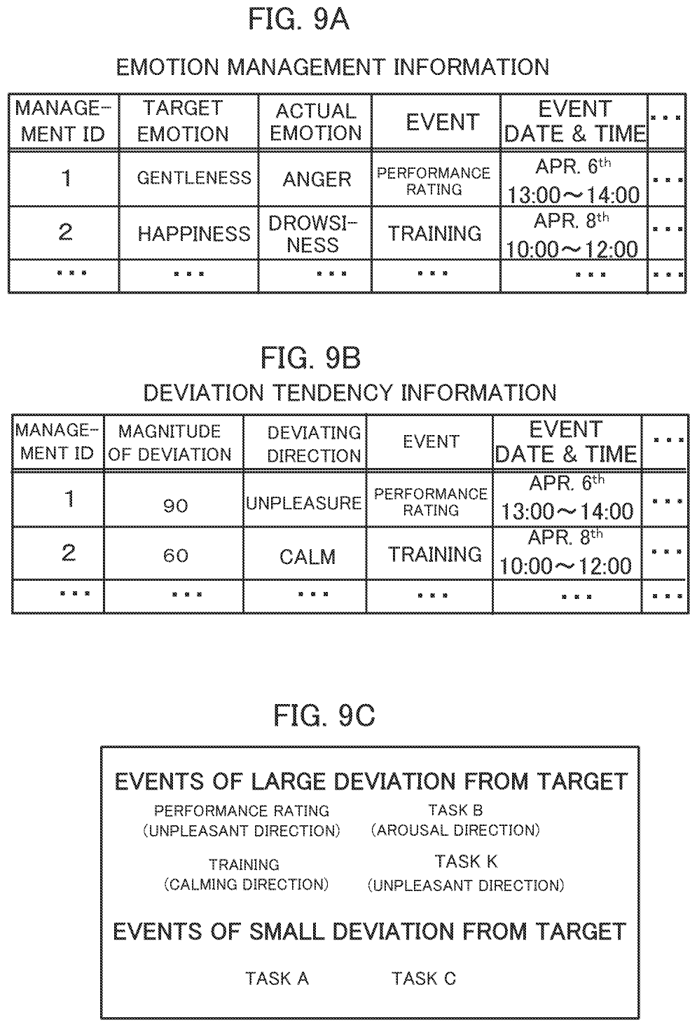

FIG. 9A is an example of a data structure of emotion management information in the second embodiment, and FIG. 9B is an example of a data structure of emotion management information in the second embodiment. The emotion control information shown in FIG. 9A is provided with items “management ID”, “target emotion”, “actual emotion”, “event”, and “event date and time”. In addition, the deviation tendency data shown in FIG. 9B is provided with items “management ID,” “deviation size”, “deviating direction”, “event”, and “event date and time”. Here, in the item “event” in the emotion management information and the deviation tendency information, the content of the event related to the corresponding target emotion and the actual emotion is registered. Besides, in the item “event date and time”, the date and time (time slot) of the event related to the corresponding target emotion and the actual emotion is registered. In the example shown in FIG. 8A and FIG. 8B, the event information indicating the content and the date and time (time slot) of the event selected from events (“personal task”, “meeting”, “drinking party”) registered in the personal schedule of the object person is registered as “event” and “event date and time” in emotion management information and deviation tendency information.

In the item “event”, the classification information indicating the class (event type) of the event specified by the user may be registered in addition to or in place of the content (details) of the event specified by the user.

FIG. 9C is an example of an emotion assessment report which the output control unit 17 causes the output device 3 to output in the second embodiment.

In the emotion assessment report shown in FIG. 9C, the output control unit 17 aggregates the deviation tendency information stored in the deviation tendency information storage unit 43 with respect to each type of the events, and outputs the deviation tendency of the emotion with respect to each type of event. In the example shown in FIG. 9C, the output control unit 17 first calculates the average vector of the deviation vectors with respect to each type of the events “performance rating”, “task B”, “training”, “task D”, “task A”, and “task C”. Then, the output control unit 17 classifies and outputs the deviation tendency of the emotion with respect to each type of the events into “events of large deviation from target” or “events of small deviation from target”, based on the average vector of the deviation vectors for each type of the events. The output control unit 17 outputs the events corresponding to “events of large deviation from target” together with information regarding the deviating direction.

In this way, in the second embodiment, the output control unit 17 aggregates deviation tendency information with respect to each type of events, and notifies the user of the aggregate result, thereby suitably supporting the recognition and management adjustment of the emotion for each event.

### (3-3) Third Embodiment

In the third embodiment, in addition to the processing in the first embodiment or the second embodiment, the information processing device 1 further acquires the degree (also referred to as “acute stress value”) of acute stress of the object person, and determines the output mode of the emotion assessment report based on the acquired acute stress value.

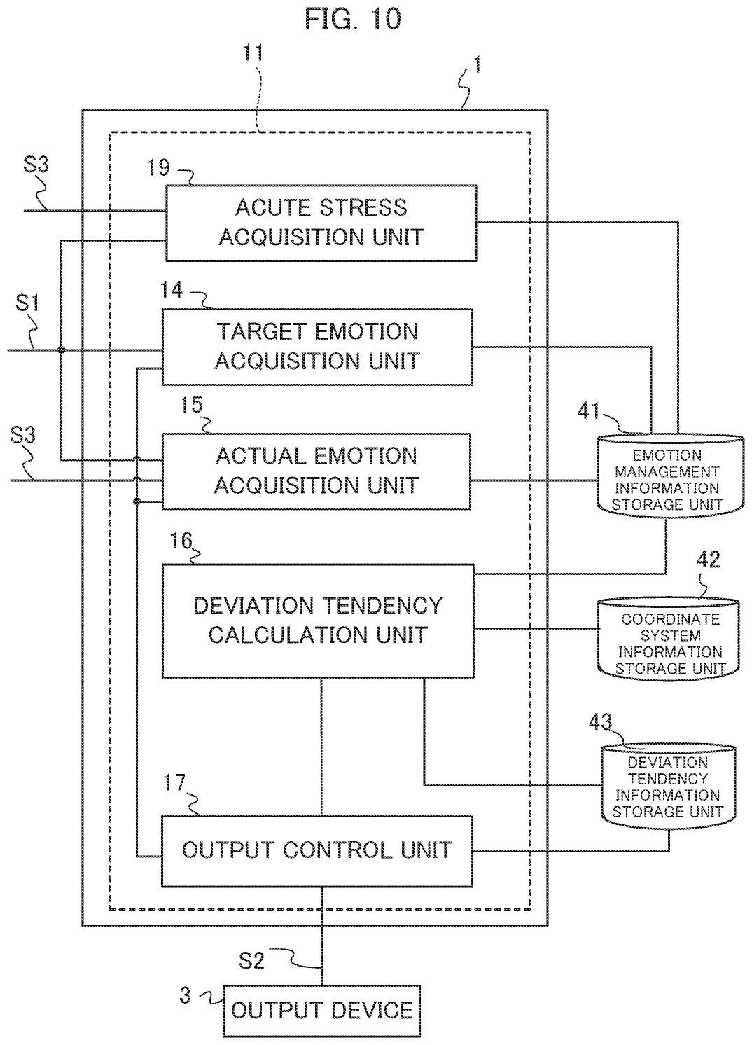

FIG. 10 is an example of functional blocks of the information processing device 1 according to the third embodiment. The processor 11 of the information processing device 1 according to the third embodiment functionally includes a target emotion acquisition unit 14, an emotion acquisition unit 15, a deviation tendency calculation unit 16, an output control unit 17, and an acute stress acquisition unit 19.

In the third embodiment, in addition to the process of acquiring the actual emotion of the object person, the information processing device 1 performs the process of acquiring the acute stress of the object person. In this case, the acute stress acquisition unit 19 calculates the acute stress value of the object person by applying any acute stress estimation method to the sensor signal S3. In this case, the acute stress acquisition unit 19 may use the biological data such as heart rate, EEG, amount of perspiration, amount of hormonal secretion, cerebral blood flow, blood pressure, body temperature, electromyogram, respiration rate, pulse wave, acceleration, etc., measured by a wearable terminal or the like worn by the object person, or may use an image obtained by photographing the face of the object person, or may use the utterance data of the object person. Then, the acute stress acquisition unit 19 stores the calculated acute stress value in the emotion management information storage unit 41 in association with the corresponding set of the actual emotion and the target emotion.

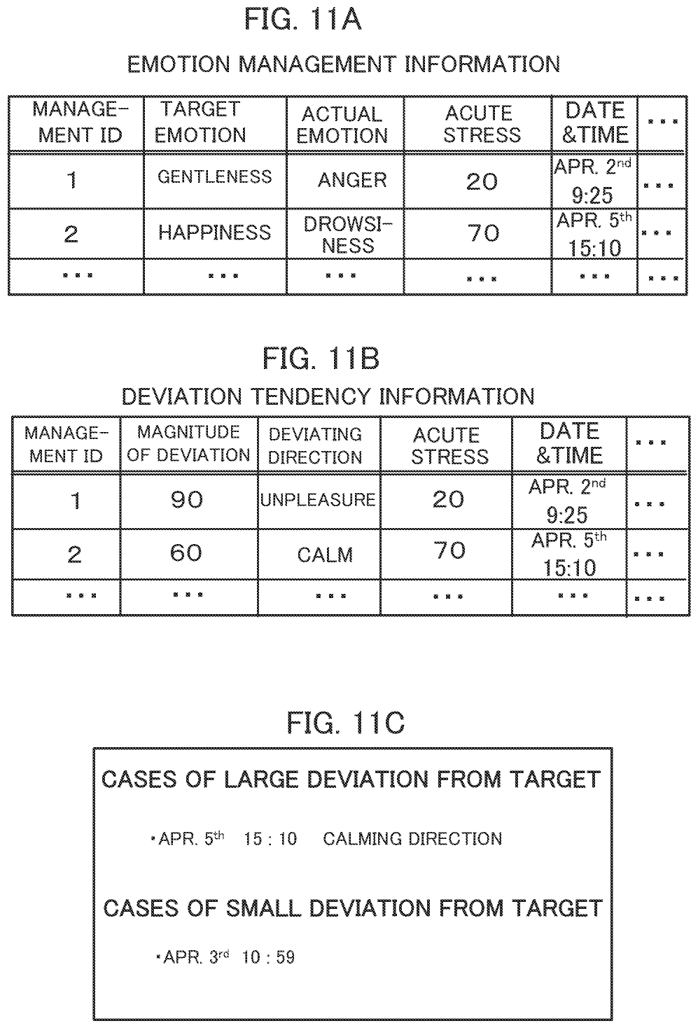

FIG. 11A is an example of a data structure of emotion control information in the third embodiment, and FIG. 11B is an example of a data structure of emotion management information in the third embodiment. As shown in FIG. 11A and FIG. 11B, in the emotion management information and the deviation tendency information, an item “acute stress” is further provided, and the acute stress values (on a scale of 0 to 100 in this case) acquired by the acute stress acquisition unit 19 are registered.

FIG. 11C is an example of an emotion assessment report that the output control unit 17 causes the output device 3 to output in the third embodiment.

In the emotion assessment report shown in FIG. 11C, the output control unit 17 selects a part of the records based on the acute stress value from the records of the deviation tendency information, and outputs the emotion assessment report based on the selected record(s) of the deviation tendency information. Specifically, the output control unit 17 selects records whose acute stress value is equal to or larger than a predetermined threshold value (e.g., 50) from the records of the deviation tendency information, and classifies and output the contents of the records into “cases of large deviation from target” or “cases of small deviation from target” based on the magnitude of deviation. For example, the output control unit 17 does not output the record corresponding to the management ID “1” shown in FIG. 11B on the emotion assessment report since the acute stress is less than the above-described threshold value. On the other hand, the output control unit 17 classifies and outputs the record corresponding to the management ID “2” shown in FIG. 11B into “cases of large deviation from target” since the acute stress is equal to or larger than the above-described threshold value.

According to this example, the output control unit 17 may present the deviation tendency information to the object person so that the priority of presenting a record increases with increasing acute stress value of the record.

The output control unit 17 may determine the display order of the records of the deviation tendency information to be outputted on the emotion assessment report based on the acute stress value, instead of selecting the records of the deviation tendency information to be outputted on the emotion assessment report based on the acute stress value. In this case, the output control unit 17 displays the deviation tendency information so that the higher the acute stress value of a record of the deviation tendency information is, the higher the display order of the record becomes on the emotion assessment report.

In yet another example, the output control unit 17 may use the acute stress value as a weight when integrating records of the deviation tendency information. In this case, for example, the deviation tendency information stored in the deviation tendency information storage unit 43 includes an item “event” as indicated in the data structure shown in FIG. 9B in the second embodiment, and with reference to records of the deviation tendency information classified with respect to each type of the events as in the second embodiment, the output control unit 17 calculates the average vector of the deviation vectors with respect to each type of the events. When calculating the above-described average vector with respect to each type of the events, the output control unit 17 uses the acute stress value as the weight for the deviation vector and performs the weighted average of the deviation vectors to calculate the above-described average vector. In this case, the output control unit 17 can suitably output an emotion assessment report in which more emphasis is placed on the deviation tendency of emotion in a condition where the acute stress value is high.

### (3-4) Fourth Embodiment

In the fourth embodiment, in addition to the processing in the second embodiment, the information processing device 1 further acquires a chronic stress value (a degree of chronic stress) of the object person, and learns a deviation tendency of the emotion in accordance with the chronic stress value.

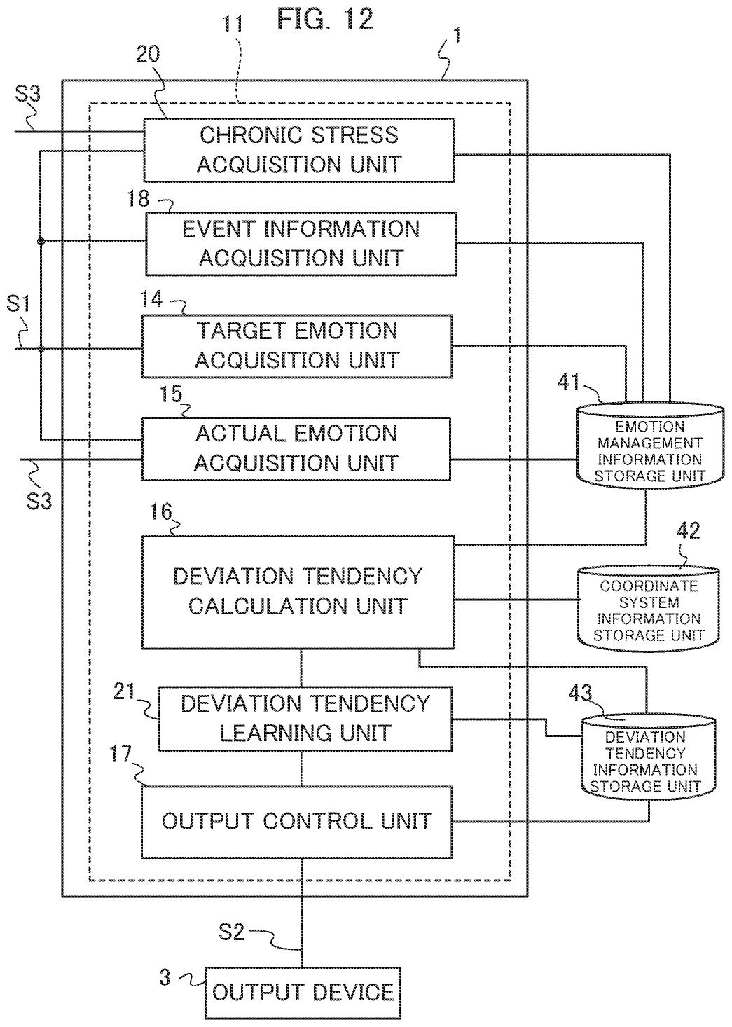

FIG. 12 is an example of functional blocks of the information processing device 1 in the fourth embodiment. The processor 11 of the information processing device 1 according to the fourth embodiment functionally includes a target emotion acquisition unit 14, an actual emotion acquisition unit 15, a deviation tendency calculation unit 16, an output control unit 17, an event information acquisition unit 18, a chronic stress acquisition unit 20, and a deviation tendency learning unit 21.

The chronic stress acquisition unit 20 calculates the chronic stress of the object person by applying a chronic stress estimation method to the sensor signal S3. In this case, the chronic stress acquisition unit 20 may use the biological data such as pulse and amount of perspiration measured by the wearable terminal or the like worn by the object person, or may use an image obtained by photographing the face of the object person, or may use the utterance data of the object person. In addition, the chronic stress acquisition unit 20 may store the sensor signal S3 periodically acquired from the object person in the storage device 4 or the memory 12 or the like and estimate the chronic stress based on the sensor signal S3 acquired within the latest predetermined period (e.g., within recent one month).

The chronic stress acquisition unit 20 stores the calculated chronic stress value in the emotion management information storage unit 41 in association with the corresponding set of actual emotion, target emotion, and event information. In addition to the items “target emotion”, “actual emotion”, and “event”, the item “chronic stress” in which the chronic stress value is recorded is included in the emotion management information stored in the emotion management information storage unit 41 and in the deviation tendency information stored in the deviation tendency information storage unit 43.

The deviation tendency learning unit 21 learns the deviation tendency of emotion in accordance with the chronic stress value and the event, based on: the information regarding the deviation vector included in the deviation tendency information stored by the deviation tendency information storage unit 43; the event information; and the chronic stress value. Specifically, the deviation tendency learning unit 21 classifies the records of the deviation tendency information by the chronic stress value, and learns the deviation tendency of emotion with respect to each type of the events for each classified record group. In this case, the deviation tendency learning unit 21 classifies the records of the deviation tendency information by both the chronic stress value and the type of the event, and calculates an average vector of the deviation vectors for each classified record group. The output control unit 17 acquires the average vector of the deviation vectors calculated for each class by the deviation tendency learning unit 21 as a learning result and outputs an emotion assessment report representing the learning result.

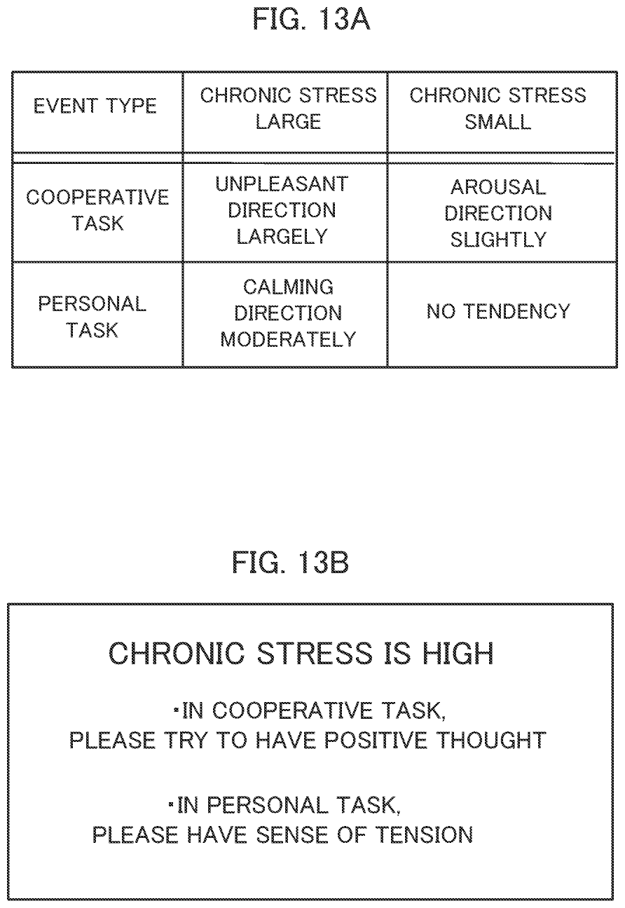

FIG. 13A is a first example of an emotion assessment report that the output control unit 17 causes the output device 3 to output in the fourth embodiment. FIG. 13B is a second example of an emotion assessment report that the output control unit 17 causes the output device 3 to output in the fourth embodiment.

In the first example, the output control unit 17 outputs a table indicating a learning result of the deviation tendency of emotion learned by the deviation tendency learning unit 21 depending on the chronic stress value and the type of the event. In the first example, there are two levels of the chronic stress value, “large chronic stress” and “small chronic stress”, and there are two categories (types) of the event, “cooperative task” and “personal task”. In this case, the deviation tendency learning unit 21 calculates the average vector of deviation vectors for each possible combination (here, 4 (=2×2) pairs) of the level of the chronic stress value and the type of the event. The output control unit 17 outputs the learning result of the deviation tendency of emotion for each combination on the emotion assessment report based on the magnitude and direction of the average vector.

In the second example, the output control unit 17 outputs the emotion assessment report illustrated in FIG. 13B if the latest chronic stress calculated by the chronic stress acquisition unit is equal to or larger than a predetermined threshold value. In this case, the output control unit 17 instructs the deviation tendency learning unit 21 to learn the deviation tendency of emotion, and the deviation tendency learning unit 21 calculates the average vector of the deviation vectors with respect to each type of events (in this case, cooperative task and personal task), on the basis of records in which the chronic stress is equal to or larger than the predetermined threshold value among the records of the deviation tendency information stored in the deviation tendency information storage unit 43. Then, the output control unit 17 outputs comment for each event type based on the average vector of the deviation vector calculated per event type. In this case, the output control unit 17 outputs comment for each event type to guide the actual emotion so that the magnitude of the corresponding average vector decreases (i.e., the actual emotion moves towards direction opposite to the average vector).

According to the fourth embodiment, the output control unit 17 suitably learns the deviation tendency of the emotion in accordance with the chronic stress value, and can suitably inform the object person or the like of the learning result. It is noted that the information processing device 1 may perform the fourth embodiment using an acute stress value instead of a chronic stress value. In this case, instead of the chronic stress acquisition unit 20, the information processing device 1 includes an acute stress acquisition unit 19, and the deviation tendency learning unit 21 learns the deviation tendency of emotion in accordance with the urgent stress value. In this case, the information processing device 1 can suitably notify the object person or the like of the learning result of the deviation tendency of emotion in accordance with the acute stress.

### (4) Processing Flow

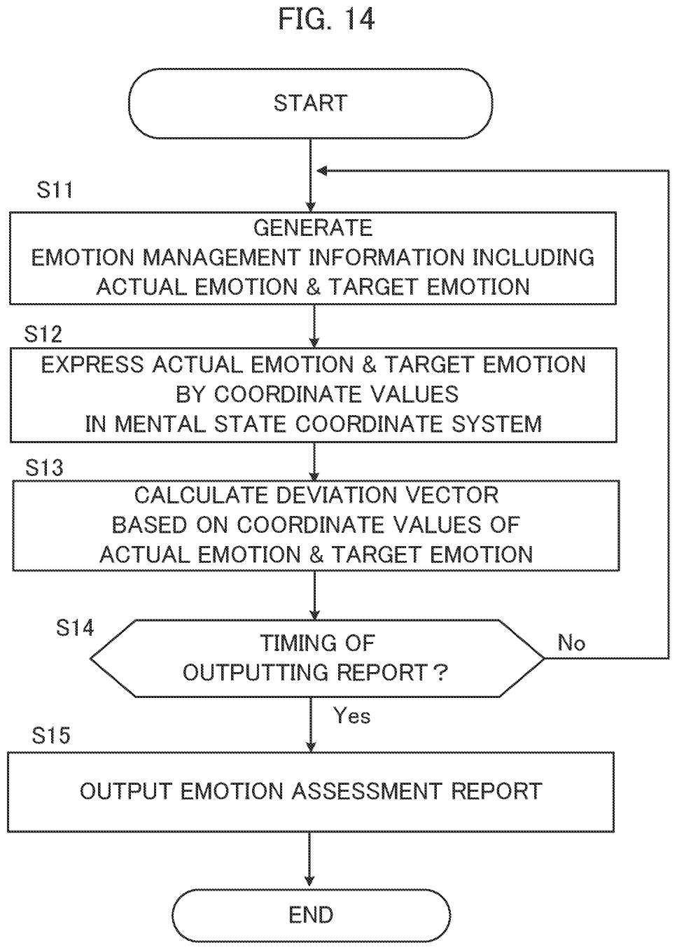

FIG. 14 is an example of a processing flowchart that is executed by the information processing device 1 according to the first example embodiment. The information processing device 1 repeatedly executes the process of the flowchart shown in FIG. 14.

First, the information processing device 1 generates emotion management information including the actual emotion and the target emotion of the object person (step S11). In this case, as illustrated in FIG. 5A, FIG. 9A, or FIG. 11A, the information processing device 1 may generate emotion management information including not only items “actual emotion” and “target emotion” but also at least any one of: the items “event” and “event date and time” indicative of content and date and time of the event; the item “chronic stress” or “acute stress” indicative of the chronic stress value or the acute stress value of the object person; and the item “date and time” indicative of the date and time when the actual emotion has come up.

Next, the information processing device 1 expresses the actual emotion and the target emotion by the coordinate values in the mental state coordinate system on the basis of the coordinate system information stored in the coordinate system information storage unit 42 (step S12). The information processing device 1 calculates the deviation vector based on the coordinate values of the actual emotion and the target emotion in the mental state coordinate system (step S13). In this case, the information processing device 1 calculates the deviation vector that is a vector in the mental state coordinate system whose start point is set to the coordinate value of the target emotion and whose end point is set to the coordinate value of the actual emotion. Then, the information processing device 1 adds a record of the deviation tendency information to be stored in the deviation tendency information storage unit 43 on the basis of the calculated deviation vector.

Then, the information processing device 1 determines whether or not it is the outputting timing of the emotion assessment report (step S14). In this case, for example, the information processing device 1 determines that it is the output timing of the emotion assessment report if a predetermined condition for outputting the emotion assessment report is satisfied or if a user's request for outputting the emotion assessment report is detected on the basis of the input signal S1.

When it is determined to be the output timing of the emotion assessment report (step S14; Yes), the information processing device 1 outputs the emotion assessment report to the output device 3 (step S15). Accordingly, the information processing device 1 can notify the object person or its manager of the deviation between the target emotion and the actual emotion of the object person, and suitably support the recognition and management adjustment of the object person's emotion. On the other hand, when it is determined not to be the output timing of the emotion assessment report (step S14; No) the information processing device 1 gets back to the process at step S11.

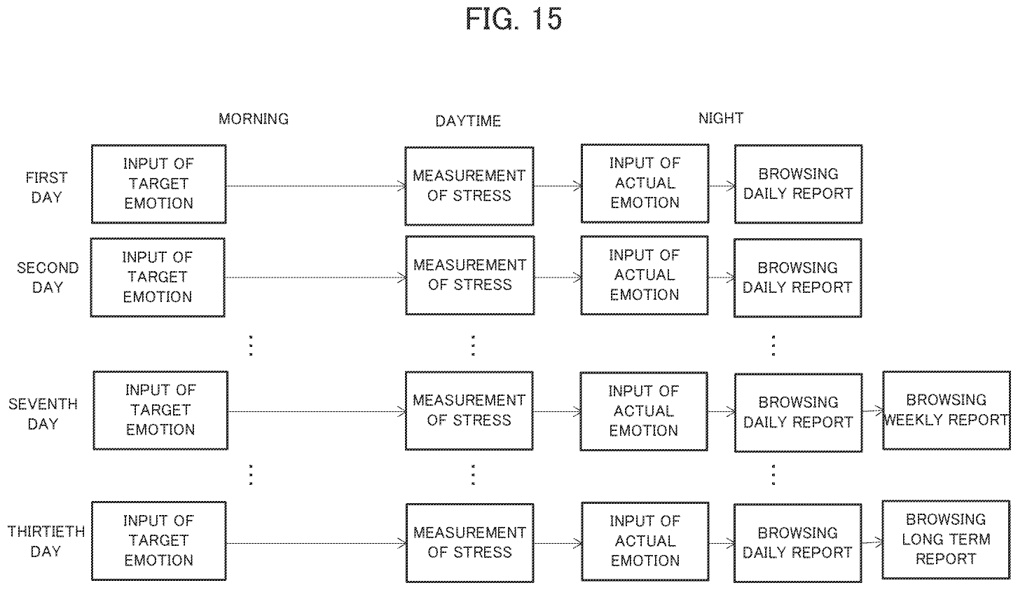

FIG. 15 shows an example of a usage scenario of the system by an object person. Here, as an example, based on the third embodiment or the fourth embodiment, the case of measuring the stress (acute stress or chronic stress) is shown.

In the usage scenario illustrated in FIG. 15, the target emotion is inputted by the input device 2 by the object person in the morning, and in the daytime, the sensor signal S3 measured by a wearable terminal or the like is supplied to the information processing device 1, so that the stress is measured. Thereafter, the object person inputs the actual emotion by the input device 2, and the information processing device 1 generates the deviation tendency information representing the deviation tendency of the emotion of the day based on the target emotion and the actual emotion inputted on the day, and outputs the emotion assessment report based on the deviation tendency information as a daily report.

In addition, every week, the information processing device 1 outputs an emotion assessment report as a weekly report based on the deviation tendency information regarding the object person generated for one week. In this case, the information processing device 1 extracts the deviation tendency information generated for one week from the deviation tendency information storage unit 43, and generates a weekly report based on the extracted deviation tendency information. Similarly, every thirty days (one month), the information processing device 1 outputs an emotion assessment report as a long-term report based on the deviation tendency information regarding the object person generated for thirty days. The information processing device 1 may evaluate the stress of the object person and output a stress alert to notify the object person that the stress is high if the stress value becomes equal to or larger than a predetermined threshold value. Further, the information processing device 1 may output an emotion assessment report (see FIG. 13B) that includes a comment as a feedback notification for guiding the actual emotion so that the magnitude of the deviation vector decreases.

Such a usage scenario allows the object person to easily grasp, manage, or adjust the emotion.

### (5) Modification

Any other device other than the information processing device 1 may be equipped with the functions of the target emotion acquisition unit 14 and the actual emotion acquisition unit 15. In this case, the other device stores the set of the target emotion and the actual emotion in the emotion management information storage unit 41 based on the input from a user. The deviation tendency calculation unit 16 of the information processing device 1 acquires the set of the target emotion and the actual emotion by referring to the emotion management information storage unit 41 and calculates the deviation vector based on the acquired set of the target emotion and the actual emotion. The output control unit 17 outputs the emotion assessment report on the basis of the deviation tendency information generated by the deviation tendency calculation unit 16. According to this mode, the information processing device 1 notifies the object person or the manager of the deviation between the target emotion and the actual emotion of the object person, and can suitably support the recognition, management, and adjustment of the emotion of the object person.

### Second Example Embodiment

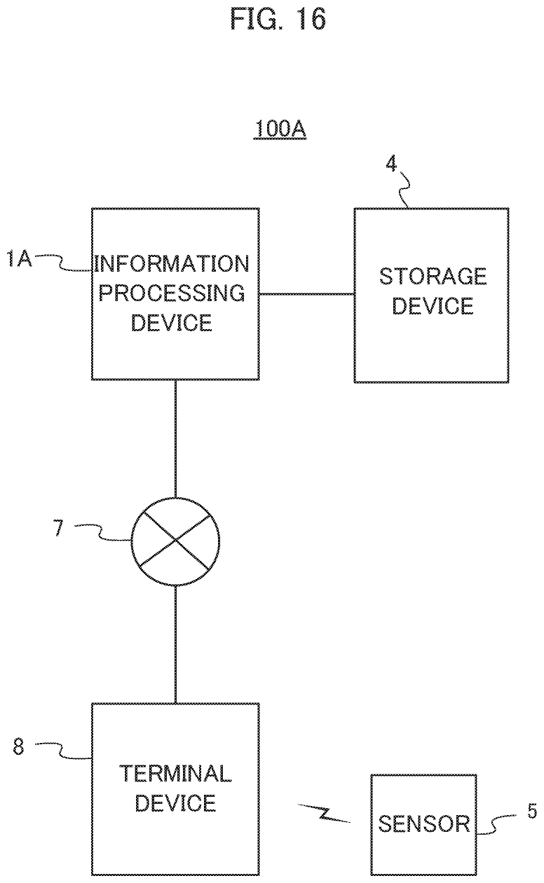

FIG. 16 shows a schematic configuration of a mental state estimation system 100A according to a second example embodiment. The mental state estimation system 100A according to the second example embodiment is a server-client model system, and an information processing device 1A functioning as a server device performs the processes which the information processing device 1 according to the first example embodiment performs. Hereinafter, the same components as those in the first example embodiment are appropriately denoted by the same reference numerals, and a description thereof will be omitted.

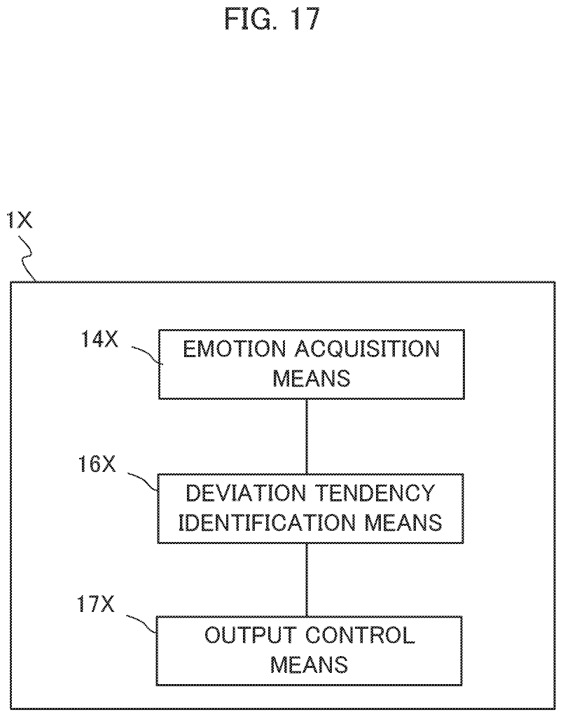

As shown in FIG. 17, the mental state estimation system 100A mainly includes an information processing device 1A that functions as a server, a storage device 4 that stores data in the same way as in the first example embodiment, and a terminal device 8 that functions as a client. The information processing device 1A and the terminal device 8 perform data communication with each other via the network 7.

The terminal device 8 is a terminal equipped with an input function, a display function, and a communication function, and functions as the input device 2 and the output device 3 shown in FIG. 1. Examples of the terminal device 8 include a personal computer, a tablet-type terminal, and a PDA (Personal Digital Assistant). The terminal device 8 transmits, to the information processing device 1A, a biological signal outputted by sensor (not shown) or an input signal based on user input.

The information processing device 1A has the same configuration as the information processing device 1 shown in FIGS. 1 to 3, for example. Then, the information processing device 1A receives information, which the information processing device 1 shown in FIG. 1 is supposed to acquire from the input device 2 and the sensor 5, from the terminal device 8 via the network 7 and identifies a deviation tendency between the target emotion and the actual emotion based on the received information. The information processing device 1A transmits the output information regarding the emotion assessment report to the terminal device 8 via the network 7 based on the request from the terminal device 8. Thus, the information processing device 1A can suitably present the deviation tendency between the target emotion and the actual emotion to the user of the terminal device 8.

### Third Example Embodiment

FIG. 17 is a block diagram of an information processing device 1X according to a third example embodiment. The information processing device 1X mainly includes an emotion acquisition means 14X, a deviation tendency identification means 16X, and an output control means 17X. The information processing device 1X may be configured by a plurality of devices.

The emotion acquisition means 14X is configured to acquire a set of target emotion, which is a target of emotion of an object person, and actual emotion, which is actual emotion of the object person. Examples of the emotion acquisition means 14X include the target emotion acquisition unit 14 and the actual emotion acquisition unit 15 in the first example embodiment (excluding the modification), and the deviation tendency calculation unit 16 in the modification.

The deviation tendency identification means 16X is configured to identify a deviation tendency between the target emotion and the actual emotion with respect to a mental state, based on the set of the target emotion and the actual emotion. Examples of the deviation tendency identification means 16X include the deviation tendency calculation unit 16 in the first example embodiment (including modification, the same hereinafter).

The output control means 17X is configured to output information regarding the deviation tendency identified by the deviation tendency identification means 16X. Examples of the output control means 17X include the output control unit 17 in the first example embodiment.

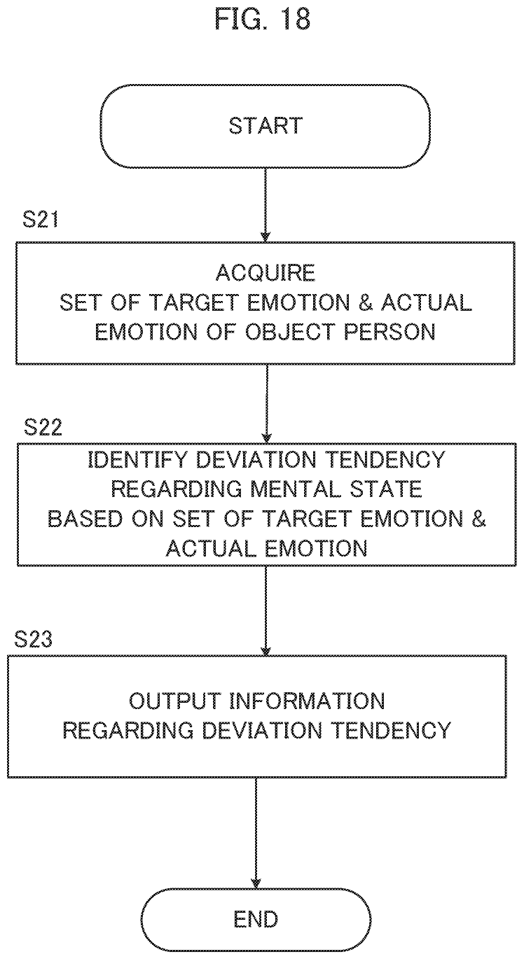

FIG. 18 is an exemplary flowchart that is executed by the information processing device 1X in the third example embodiment. The emotion acquisition means 14X acquires a set of target emotion, which is a target of emotion of an object person, and actual emotion, which is actual emotion of the object person (step S21). The deviation tendency identification means 16X identifies a deviation tendency between the target emotion and the actual emotion with respect to a mental state, based on the set of the target emotion and the actual emotion (step S22). The output control means 17X outputs information regarding the deviation tendency identified by the deviation tendency identification means 16X (step S23).

The information processing device 1X according to the third example embodiment can notify the object person or the manager of the deviation between the target emotion and the actual emotion of the object person, and suitably support understanding, managing, and adjusting the object person's emotion.

In the example embodiments described above, the program is stored by any type of a non-transitory computer-readable medium (non-transitory computer readable medium) and can be supplied to a control unit or the like that is a computer. The non-transitory computer-readable medium include any type of a tangible storage medium. Examples of the non-transitory computer readable medium include a magnetic storage medium (e.g., a flexible disk, a magnetic tape, a hard disk drive), a magnetic-optical storage medium (e.g., a magnetic optical disk), CD-ROM (Read Only Memory), CD-R, CD-R/W, a solid-state memory (e.g., a mask ROM, a PROM (Programmable ROM), an EPROM (Erasable PROM), a flash ROM, a RAM (Random Access Memory)). The program may also be provided to the computer by any type of a transitory computer readable medium. Examples of the transitory computer readable medium include an electrical signal, an optical signal, and an electromagnetic wave. The transitory computer readable medium can provide the program to the computer through a wired channel such as wires and optical fibers or a wireless channel.

The whole or a part of the example embodiments (including modifications, the same shall apply hereinafter) described above can be described as, but not limited to, the following Supplementary Notes.

[Supplementary Note 1]

An information processing device comprising:
an emotion acquisition means configured to acquire a set of target emotion, which is a target of emotion of an object person, and actual emotion, which is actual emotion of the object person;
a deviation tendency identification means configured to identify a deviation tendency between the target emotion and the actual emotion with respect to a mental state, based on the set of the target emotion and the actual emotion; and
an output control means configured to output information regarding the deviation tendency.

[Supplementary Note 2]

The information processing device according to Supplementary Note 1,
wherein the deviation tendency identification means is configured to
   express the target emotion and the actual emotion by coordinate values in a coordinate system regarding the mental state, and
   identify the deviation tendency based on a vector specified by the coordinate value of the target emotion and the coordinate value of the actual emotion.

[Supplementary Note 3]

The information processing device according to Supplementary Note 2,
wherein the output control means is configured to determine an output mode of the information regarding the deviation tendency based on magnitude of the vector.

[Supplementary Note 4]

The information processing device according to any one of Supplementary Notes 1 to 3, further comprising
an event information acquisition means configured to acquire event information related to the object person,
wherein the output control means is configured to determine an output mode of the information regarding the deviation tendency based on the event information.

[Supplementary Note 5]

The information processing device according to Supplementary Note 4,
wherein the output control means is configured to output the information regarding the deviation tendency for each type of events indicated by the event information.

[Supplementary Note 6]

The information processing device according to any one of Supplementary Notes 1 to 5, further comprising
a stress acquisition means configured to acquire a degree of stress of the object person for each set of the target emotion and the actual emotion,
wherein the output control means is configured to determine an output mode of information regarding the deviation tendency, based on the + degree of the stress.

[Supplementary Note 7]

The information processing device according to Supplementary Note 6,
wherein the output control means is configured to increase priority of outputting the deviation tendency regarding a set of the target emotion and the actual emotion, with increasing degree of acute stress of the object person regarding the set of the target emotion and the actual emotion.

[Supplementary Note 8]

The information processing device according to Supplementary Note 6, further comprising
a deviation tendency learning means configured to
   classify the set of the target emotion and the actual emotion based on a degree of chronic stress of the object person and
   learn the deviation tendency for each class,
wherein the output control means is configured to output a learning result of the deviation tendency by the deviation tendency learning means.

[Supplementary Note 9]

The information processing device according to Supplementary Note 6, further comprising
a deviation tendency learning means configured to
   classify the set of the target emotion and the actual emotion based on a degree of acute stress of the object person and
   learn the deviation tendency for each class,
wherein the output control means is configured to output a learning result of the deviation tendency by the deviation tendency learning means.

[Supplementary Note 10]

The information processing device according to any one of Supplementary Notes 1 to 9,
wherein the emotion acquisition means is configured to acquire the target emotion and the actual emotion by receiving an external input that specifies the target emotion and the actual emotion.

[Supplementary Note 11]

A control method executed by a computer, the control method comprising:
acquiring a set of target emotion, which is a target of emotion of an object person, and actual emotion, which is actual emotion of the object person;
identifying a deviation tendency between the target emotion and the actual emotion with respect to a mental state, based on the set of the target emotion and the actual emotion; and
outputting information regarding the deviation tendency.

[Supplementary Note 12]

A storage medium storing a program executed by a computer, the program causing the computer to:
acquire a set of target emotion, which is a target of emotion of an object person, and actual emotion, which is actual emotion of the object person;
identify a deviation tendency between the target emotion and the actual emotion with respect to a mental state, based on the set of the target emotion and the actual emotion; and
output information regarding the deviation tendency.

While the invention has been particularly shown and described with reference to example embodiments thereof, the invention is not limited to these example embodiments. It will be understood by those of ordinary skilled in the art that various changes in form and details may be made therein without departing from the spirit and scope of the present invention as defined by the claims. In other words, it is needless to say that the present invention includes various modifications that could be made by a person skilled in the art according to the entire disclosure including the scope of the claims, and the technical philosophy. All Patent and Non-Patent Literatures mentioned in this specification are incorporated by reference in its entirety.

### DESCRIPTION OF REFERENCE NUMERALS

1, 1A, 1X Information processing device
2 Input device
3 Output device
4 Storage device
5 Sensor
8 Terminal device
100, 100A Mental state estimation system

What is claimed is:

1. An information processing device comprising:
at least one memory configured to store instructions; and
at least one processor configured to execute the instructions to:
acquire a set of target emotion, which is a target of emotion of an object person, and actual emotion, which is actual emotion of the object person;
identify a deviation tendency between the target emotion and the actual emotion with respect to a mental state, based on the set of the target emotion and the actual emotion; and
output information regarding the deviation tendency.

2. The information processing device according to claim 1,
wherein the at least one processor is configured to execute the instructions to
express the target emotion and the actual emotion by coordinate values in a coordinate system regarding the mental state, and
identify the deviation tendency based on a vector specified by the coordinate value of the target emotion and the coordinate value of the actual emotion.

3. The information processing device according to claim 2,
wherein the at least one processor is configured to execute the instructions to determine an output mode of the information regarding the deviation tendency based on magnitude of the vector.

4. The information processing device according to claim 1,
wherein the at least one processor is configured to further execute the instructions to acquire event information related to the object person, and
wherein the at least one processor is configured to execute the instructions to determine an output mode of the information regarding the deviation tendency based on the event information.

5. The information processing device according to claim 4,
wherein the at least one processor is configured to execute the instructions to output the information regarding the deviation tendency for each type of events indicated by the event information.

6. The information processing device according to claim 1,
wherein the at least one processor is configured to further execute the instructions to acquire a degree of stress of the object person for each set of the target emotion and the actual emotion, and
wherein the output control means is configured at least one processor is configured to execute the instructions to determine an output mode of information regarding the deviation tendency, based on the degree of the stress.

7. The information processing device according to claim 6,
wherein the at least one processor is configured to execute the instructions to increase priority of outputting the deviation tendency regarding a set of the target emotion and the actual emotion, with increasing degree of acute stress of the object person regarding the set of the target emotion and the actual emotion.

8. The information processing device according to claim 6,
wherein at least one processor is configured to further execute the instructions to
classify the set of the target emotion and the actual emotion based on a degree of chronic stress of the object person and
learn the deviation tendency for each class, and
wherein the at least one processor is configured to execute the instructions to output a learning result of the deviation tendency.

9. The information processing device according to claim 6,
wherein the at least one processor is configured to further execute the instructions to
classify the set of the target emotion and the actual emotion based on a degree of acute stress of the object person and
learn the deviation tendency for each class, and
wherein the at least one processor is configured to execute the instructions to output a learning result of the deviation tendency.

10. The information processing device according to claim 1,
wherein the at least one processor is configured to execute the instructions to acquire the target emotion and the actual emotion by receiving an external input that specifies the target emotion and the actual emotion.

11. A control method executed by a computer, the control method comprising:
acquiring a set of target emotion, which is a target of emotion of an object person, and actual emotion, which is actual emotion of the object person;
identifying a deviation tendency between the target emotion and the actual emotion with respect to a mental state, based on the set of the target emotion and the actual emotion; and
outputting information regarding the deviation tendency.

12. A non-transitory computer readable storage medium storing a program executed by a computer, the program causing the computer to:
acquire a set of target emotion, which is a target of emotion of an object person, and actual emotion, which is actual emotion of the object person;
identify a deviation tendency between the target emotion and the actual emotion with respect to a mental state, based on the set of the target emotion and the actual emotion; and
output information regarding the deviation tendency.
